(19) United States
(12) Patent Application Publication
(10) Pub. No.: US 2009/0240061 A1
(43) Pub. Date: Sep. 24, 2009

(54) Method for forming a carbon-carbon or carbon-heteroatom bond

(75) Inventors: Jean-Francis Spindler, Ternay (FR); Marc Taillefer, Vaihauques (FR); Henri-Jean Cristau, Saint-Aunes (FR); Pascal Philippe Cellier, Paris (FR); Armelle Ouali, Beaumont (FR)

Correspondence Address: STITES & HARBISON PLLC, 1199 NORTH FAIRFAX STREET, SUITE 900, ALEXANDRIA, VA, 22314, US

(73) Assignee: Shasun Pharma Solutions, Inc., Cranbury, NJ (US)

(21) Appl. No.: 10/568,850

(22) PCT Filed: Jul. 28, 2004
(86) PCT No.: PCT/FR2004/002021

§ 371 Date: May 10, 2007

(30) Foreign Application Priority Data
Aug. 28, 2003 (FR) 03 10253

(51) Int. Cl. C07D 231/12; C07C 41/01; C07C 67/00

(52) U.S. Cl. 548/377.1; 568/659; 560/81

(57) ABSTRACT

The invention relates to a method for forming a carbon-carbon or carbon-heteroatom bond by reacting an unsaturated compound carrying a leaving group and a nucleophilic compound, in the absence of a ligand. The aim of the invention is especially to form carbon-nitrogen bonds according to a method for the arylation of nitrogenated organic derivatives. According to the inventive method, a carbon-carbon or carbon-heteroatom bond is formed by reacting an unsaturated compound carrying a leaving group with a nucleophilic compound donating a carbon atom or a heteroatom (HE) that can substitute the leaving group, thus forming a C—C or C—HE bond, in the presence of a copper-based catalyst and a base. Said metal is characterised in that the reaction takes place in the absence of a ligand and in a nitrile-type solvent.

The subject of the present invention is a method for creating a carbon-carbon or carbon-heteroatom bond by the reaction of an unsaturated compound carrying a leaving group and a nucleophilic compound, in the absence of a ligand.

The invention relates in particular to the creation of a carbon-nitrogen bond according to a method for the arylation of nitrogen-containing organic derivatives.

There are numerous major compounds which are used in the agrochemical and pharmaceutical fields, for example arylhydrazines, which result from the arylation of a nucleophilic compound by the creation of a carbon-nitrogen bond.

A conventional method of arylation consists in using the Ullmann reaction (Ullmann F. and Kipper H., Ber. dtsch. Chem. Ges. 1905, 38, 2120-2126), by the prolonged heating of reagents at high temperature, in the presence of catalytic or stoichiometric copper. The reactions are often limited to the use of aryl iodides and their yields are reduced by the competitive formation of products of biaryl homocoupling.

Arylation reactions involve a catalyst and several types of catalysts have been described.

Palladium was used by Buchwald et al. to carry out in particular the reaction for arylation of indoles (Org. Lett. 2000, 2, 1403-1406), in the presence of a base, in toluene at 80° C.-100° C. Generally, the yields are satisfactory but the reaction temperature remains nevertheless high for this type of palladium-based catalyst.

Copper has also been used (Chiriac et al., Rev. Roum. Chim. 1969, 14, 1263-1267) to carry out the arylation of sodium salts of pyrazoles by iodobenzene in the presence of a catalytic quantity of copper, under refluxing DMF. The conditions described are very hard, the temperature is 153° C. and the reaction time is very long, from 30 to 40 hours.

Beletskaya et al. (Tetrahedron Lett. 1998, 39, 5617-5622) have proposed to combine palladium with copper in the case of the N-arylation of benzotriazole. The presence of copper is essential in order to control the selectivity of the reaction. The catalysis is a phase-transfer catalysis which is not easy to carry out on an industrial scale.

Moreover, it has been proposed, according to WO 98/00399, to use a nickel catalyst, but the latter has proved scarcely effective for carrying out the arylation of heterocycles such as imidazole.

Chen et al. have also described (J. Chem. RES. (S) 2000, 367-369), the arylation of azoles from diaryliodonium salts, in the presence of a cobalt catalyst, under phase-transfer conditions.

Buchwald et al. (J. Am. Chem. Soc. 2001, 123, 7727-7729) have recently developed a method for the arylation of nitrogen-containing nucleophilic compounds catalyzed by copper. Its catalytic system, composed of a catalyst which is insensitive to air, copper(I) iodide and trans-1,2-diaminocyclohexane ligand, allows the arylation, in dioxane at 110° C., of heterocycles such as pyrazoles, indoles, carbazole, pyrrole, indazole, imidazole, phthalazinone and 7-azaindole.

It is evident from an examination of the state of the art that up until now, copper has always been combined with a ligand.

The objective of the present invention is to provide a method which overcomes the abovementioned disadvantages and which makes it possible to deal with a very large number of nucleophilic compounds.

More specifically, the subject of the present invention is a method for creating a carbon-carbon or carbon-heteroatom bond by the reaction of an unsaturated compound carrying a leaving group and a nucleophilic compound donating a carbon atom or a heteroatom (HE) capable of substituting for the leaving group, thus creating a C—C or C—HE bond, in the presence of a copper-based catalyst and a base, characterized in that the reaction takes place in the absence of a ligand and in a nitrile-type solvent.

It has now been found that it is possible to carry out the coupling between an unsaturated substrate and a nucleophilic compound in the absence of a ligand as long as the reaction is carried out in a nitrile-type solvent, preferably acetonitrile.

According to a first variant of the method of the invention, an arylation reaction is carried out by reacting an aromatic compound carrying a leaving group and a nucleophilic compound.

According to another variant of the method of the invention, a vinylation reaction is carried out by reacting, respectively, a compound having a double bond at the α-position with respect to a leaving group and a nucleophilic compound.

In the text which follows in the present invention, the term “arylation” is used in a broad sense since the use of an unsaturated compound carrying a leaving group which is either of the unsaturated aliphatic type, or of the carbocyclic or heterocyclic aromatic type, is envisaged.

The expression “nucleophilic compound” is understood to mean an organic hydrocarbon compound which may be acyclic or cyclic and whose characteristic is to comprise at least one atom carrying a free doublet which may or may not comprise a charge, and preferably a nitrogen, oxygen, phosphorus or carbon atom.

As mentioned above, the nucleophilic compound comprises at least one atom carrying a free doublet which may be donated by a functional group and/or a carbanion.

As functional groups comprising said atoms and/or carbanions, the following atoms and groups may be mentioned in particular:

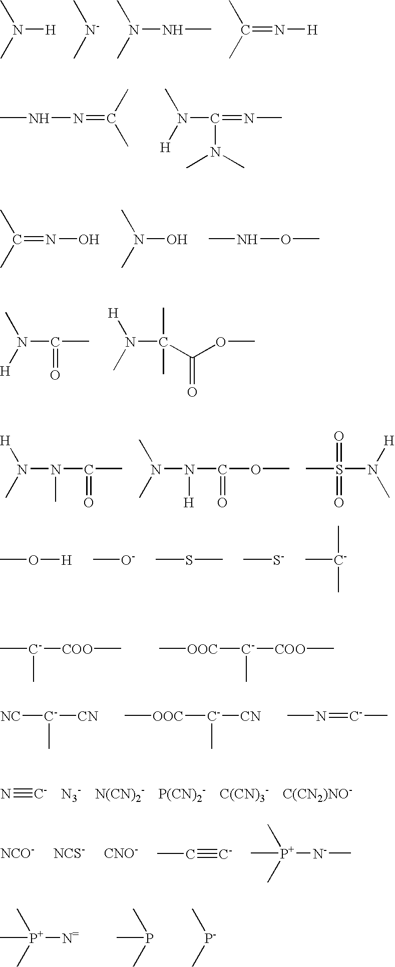

According to another variant of the invention, the nucleophilic compound comprises at least one nitrogen atom carrying a free doublet contained in a saturated, unsaturated or aromatic ring: the ring generally comprising from 3 to 8 atoms.

It should be noted that when the nucleophilic compound comprises a functional group, of which examples are given above, which carries one or two negative charges, said compound is then in a solidified form. The counter-ion is generally a metal cation such as an alkali metal, preferably sodium or lithium, an alkaline-earth metal, preferably calcium, or the residue of an organometallic compound such as in particular a magnesium or zinc compound.

### Nucleophilic Compounds

The method of the invention involves a large number of nucleophilic compounds and examples are given below, by way of illustration and with no limitation being implied.

A first category of substrates to which the method of the invention applies are the primary or secondary amines and the imines.

More specifically, the primary or secondary amines may be represented by a general formula:

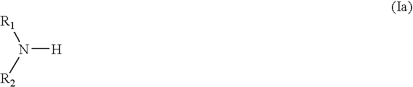

in said formula (Ia):
R₁ and R₂ represent, independently of each other, a hydrogen atom or a hydrocarbon group having from 1 to 20 carbon atoms which may be a saturated or unsaturated, linear or branched acyclic aliphatic group; a monocyclic or polycyclic, saturated, unsaturated or aromatic carbocyclic or heterocyclic group; a succession of the abovementioned groups,
at most one of the groups R₁ and R₂ represents a hydrogen atom.

In formula (Ia), the various symbols may have more particularly the meaning given below.

Thus, R₁ and R₂ may represent, independently of each other, a saturated or unsaturated, linear or branched acyclic aliphatic group.

More specifically, R₁ and R₂ preferably represent a saturated, linear or branched acyclic aliphatic group, preferably as C₁ to C₁₂, and more preferably still as C₁ to C₄.

The invention does not exclude the presence of an unsaturation in the hydrocarbon chain, such as one or more double bonds which may be conjugated or unconjugated.

The hydrocarbon chain may be optionally interrupted by a heteroatom (for example oxygen, sulfur, nitrogen or phosphorus) or by a functional group insofar as the latter does not react, and there may be mentioned in particular a group such as in particular —CO—.

The hydrocarbon chain may optionally carry one or more substituents (for example halogen, ester, amino or alkyl and/or arylphosphine) insofar as there is no interference.

The saturated or unsaturated, linear or branched acyclic aliphatic group may optionally carry a cyclic substituent. The expression ring is understood to mean a saturated, unsaturated or aromatic carbocyclic or heterocyclic ring.

The acyclic aliphatic group may be attached to the ring by a valency bond, a heteroatom or a functional group such as oxy, carbonyl, carboxyl, sulfonyl and the like.

As examples of cyclic substituents, it is possible to envisage aromatic or heterocyclic cycloaliphatic substituents, in particular cycloaliphatic substituents comprising 6 carbon atoms in the ring or benzene substituents, these cyclic substituents themselves optionally carrying any substituent insofar as they do not hamper the reactions involved in the method of the invention. C₁ to C₄ alkoxy or alkyl groups may be mentioned in particular.

Among the aliphatic groups carrying a cyclic substituent, reference may be made more particularly to cycloalkylalkyl, for example cyclohexylalkyl, groups, or preferably C₇ to C₁₂ arylalkyl, in particular benzyl or phenylethyl, groups.

In general formula (Ia), the groups R₁ and R₂ may also represent, independently of each other, a saturated carbocyclic group comprising one or two unsaturations in the ring, generally as C₃ to C₈, preferably having 6 carbon atoms in the ring; it being possible for said ring to be substituted. As preferred examples of this type of groups, there may be mentioned cyclohexyl groups optionally substituted with linear or branched alkyl groups, having from 1 to 4 carbon atoms.

The groups R₁ and R₂ may represent, independently of each other, an aromatic hydrocarbon, and in particular a benzene, group corresponding to general formula (F₁):

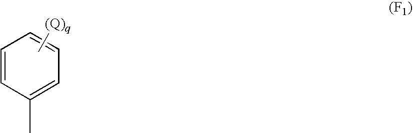

in which:
q represents an integer from 0 to 5,
Q represents a group chosen from a linear or branched C₁ to C₆ alkyl group, a linear or branched C₁ to C₆ alkoxy group, a linear or branched C₁ to C₆ alkylthio group, an —NO₂ group, a —CN group, a halogen atom, a CF₃ group.

R₁ and R₂ may also represent, independently of each other, a polycyclic aromatic hydrocarbon group with rings which can form with each other ortho-fused, ortho- and peri-fused systems. There may also be mentioned, more particularly, a naphthyl group; it being possible for said ring to be substituted.

R₁ and R₂ may also represent, independently of each other, a polycyclic hydrocarbon group consisting of at least 2 saturated and/or unsaturated carbocycles or of at least 2 carbocycles of which at least one is aromatic, and forming with each other ortho- or ortho- and peri-fused systems. Generally, the rings are C₃ to C₈, preferably C₆. As more particular examples, there may be mentioned the bornyl group or the tetrahydronaphthalene group.

R₁ and R₂ may also represent, independently of each other, a saturated, unsaturated or aromatic heterocyclic group containing in particular 5 or 6 atoms in the ring, of which one or two heteroatoms such as nitrogen (not substituted with a hydrogen atom), sulfur and oxygen atoms; it being possible for the carbon atoms of this heterocycle to also be substituted.

R₁ and R₂ may also represent a polycyclic heterocyclic group defined as being either a group consisting of at least 2 aromatic or nonaromatic heterocycles containing at least one heteroatom in each ring and forming with each other ortho- or ortho- and peri-fused systems, or a group consisting of at least one aromatic or nonaromatic hydrocarbon ring and at least one aromatic or nonaromatic heterocycle forming with each other ortho- or ortho- and peri-fused systems; it being possible for the carbon atoms of said rings to be optionally substituted.

By way of examples of R₁ and R₂ groups of the heterocyclic type, there may be mentioned, inter alia, furyl, thienyl, isoxazolyl, furazanyl, isothiazolyl, pyridyl, pyridazinyl, pyrimidinyl, pyranyl, phosphino groups and quinolyl, naphthyridinyl, benzopyranyl and benzofuranyl groups.

The number of substituents present on each ring depends on the carbon condensation of the ring and the presence or absence of unsaturation in the ring. The maximum number of substituents which may be carried by a ring is easily determined by a person skilled in the art.

The amines used preferably correspond to formula (Ia) in which R₁ and R₂, which are identical or different, represent a C₁ to C₁₅, preferably C₁ to C₁₀, alkyl group, a C₃ to C₈, preferably C₅ to C₆, cycloalkyl group, a C₆ to C₁₂ aryl or arylalkyl group.

As more particular examples of R₁ and R₂ groups, there may be mentioned the C₁ to C₄ alkyl, phenyl, benzyl or naphthyl groups.

As more specific examples of amines corresponding to formula (Ia), there may be mentioned aniline, N-methyl-aniline, diphenylamine, benzylamine and dibenzylamine.

As regards the imines, they may be represented by the following formula:

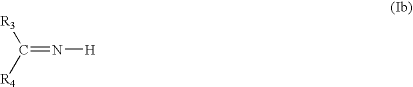

in said formula:
R₃ and R₄, which are identical or different, have the meaning given for R₁ and R₂ in formula (Ia),
at most one of the groups R₃ and R₄ represents a hydrogen atom.

The imines used preferably correspond to formula (Ib) in which R₃ and R₄, which are identical or different, represent a C₁ to C₁₅, preferably C₁ to C₁₀, alkyl group, a C₃ to C₈, preferably C₅ or C₆, cycloalkyl group, a C₆ to C₁₂ aryl or arylalkyl group.

As more particular examples of groups R₃ and R₄, there may be mentioned the C₁ to C₄ alkyl, phenyl, benzyl or naphthyl groups.

Other nucleophiles which may be used in the method of the invention are oximes and hydroxylamines.

The oximes may be represented by the following formula:

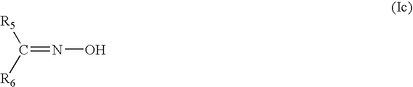

in said formula:
R₅ and R₆, which are identical or different, have the meaning given for R₁ and R₂ in formula (Ia),
at most one of the groups R₅ and R₆ represents a hydrogen atom.

The hydroxyamines may be represented by the following formula:

R₇—NH—OR₈ (Id)

in said formula:
R₇ has the meaning given for R₁ and R₂ in formula (Ia) with the exception of a hydrogen atom,
R₈ represents a hydrogen atom, a saturated or unsaturated, linear or branched acyclic aliphatic group; a monocyclic or polycyclic, saturated or unsaturated carbocyclic group; a succession of the abovementioned groups.

The oximes or hydroxylamines preferably used correspond to the formulae (Ic) or (Id) in which R₅, R₆ and R₇ represent more particularly a C₁ to C₁₅, preferably C₁ to C₁₀, alkyl group, a C₃ to C₈, preferably C₅ or C₆, cycloalkyl group, a C₆ to C₁₂ aryl or arylalkyl group.

As more particular examples of groups R₅, R₆ and R₇, there may be mentioned C₁ to C₄ alkyl, phenyl, benzyl or naphthyl groups.

As regards R₈, it is preferably a C₁ to C₄ alkyl group or a benzyl group.

The invention relates more particularly to the nucleophilic compounds of hydrazine-type. They may be symbolized by the following formula:

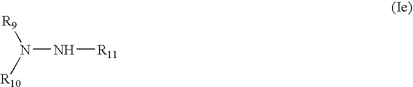

in said formula:
R₉, R₁₀ and R₁₁, which are identical or different, have the meaning given for R₁ and R₂ in formula (Ia),
R₁₁ represents a hydrogen atom or a protecting group G,
at least one of the groups R₉, R₁₀ or R₁₁ is not a hydrogen atom,
or alternatively R₉ and R₁₀ may be linked so as to constitute, with the carbon atoms carrying them, a monocyclic or polycyclic, saturated, unsaturated or aromatic carbocyclic or heterocyclic group having from 3 to 20 atoms.

The hydrazines preferably used correspond to the formula (Ia) in which R₉ and R₁₀, which are identical or different, represent a C₁ to C₁₅, preferably C₁ to C₁₀, alkyl group, a C₃ to C₈, preferably C₅ or C₆, cycloalkyl group, a C₆ to C₁₂ aryl or arylalkyl group.

As more particular examples of groups R₉ and R₁₀, there may be mentioned the C₁ to C₄ alkyl, phenyl, benzyl or naphthyl groups.

R₉ and R₁₀ may be linked so as to constitute, with the carbon atoms carrying them, a monocyclic or polycyclic, saturated, unsaturated or aromatic carbocyclic or heterocyclic group having from 3 to 20 atoms, comprising two or three ortho-fused rings, which means that at least two rings have two carbon atoms in common. In the case of the polycyclic compounds, the number of atoms in each ring preferably varies between 3 and 6. R₉ and R₁₀ preferably form a cyclohexane or fluorenone type ring.

In formula (Ie), R₁₁ more particularly represents a hydrogen atom, an alkyl group, preferably as C₁ to C₁₂; an alkenyl or alkynyl group, preferably as C₂ to C₁₂; a cycloalkyl group, preferably as C₃ to C₁₂; an aryl or arylalkyl group, preferably as C₆ to C₁₂.

R₁₁ preferably represents a hydrogen atom or a C₁-C₄ alkyl group.

It should be noted that when the nucleophilic substrate comprises an NH₂ group in which the two hydrogen atoms are capable of reacting, it is possible, in order to improve the selectivity of the reaction, to block one of them, with the aid of a protecting group. Use is made of the protecting groups commonly used for this purpose, and mention may be made in particular of groups such as acyl (acetyl, benzoyl), BOC (butyloxycarbonyl), CBZ (carbobenzoxy), FMOC (trifluoromethyloxycarbonyl) or MSOC (methanesulfenyl-2-ethoxycarbonyl).

Reference may be made to the text book by Theodora W. Greene et al., Protective Groups in Organic Synthesis, (2nd edition) John Wiley & Sons, Inc to carry out the protection of the amino group and its deprotection.

As other types of nucleophilic substrates, hydrazones may be mentioned. They may be represented by the following formula:

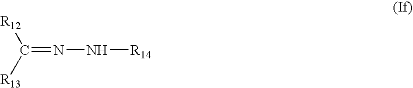

in said formula:
R₁₂, R₁₃ and R₁₄, which are identical or different, have the meaning given for R₁ and R₂ in formula (Ia),
at most one of the groups R₁₂ and R₁₃ represents a hydrogen atom,
or alternatively, R₁₂ and R₁₃ may be linked so as to constitute, with the carbon atoms carrying them, a monocyclic or polycyclic, saturated, unsaturated or aromatic carbocyclic or heterocyclic group having from 3 to 20 atoms.

The hydrazones preferably used correspond to formula (If), in which R₁₂ and R₁₃, which are identical or different, represent a C₁ to C₁₅, preferably C₁ to C₁₀, alkyl group, a C₃ to C₈, preferably C₅ or C₆, cycloalkyl group, a C₆ to C₁₂ aryl or arylalkyl group.

As more particular examples of groups R₁₂ and R₁₃, there may be mentioned the C₁ to C₄ alkyl, phenyl, benzyl or naphthyl groups.

R₁₂ and R₁₃ may be linked so as to constitute, with the carbon atoms carrying them, a monocyclic or polycyclic, saturated, unsaturated or aromatic carbocyclic or heterocyclic group having from 3 to 20 carbon atoms, comprising two or three ortho-fused rings. In the case of the polycyclic compounds, the number of atoms in each ring preferably varies between 3 and 6. R₁₂ and R₁₃ preferably form a cyclohexane or fluorenone type ring.

In formula (If), R₁₄ more particularly represents a hydrogen atom, an alkyl group, preferably as C₁ to C₁₂; an alkenyl or alkynyl group, preferably as C₂ to C₁₂; a cycloalkyl group, preferably as C₃ to C₁₂; an aryl or arylalkyl group, preferably as C₆ to C₁₂.

R₁₄ preferably represents a hydrogen atom or a C₁-C₄ alkyl group.

The invention also relates to the amide-type compounds corresponding more particularly to formula (Ig):

R₁₅—NH—CO—R₁₆ (Ig)

in said formula (Ig), R₁₅ and R₁₆ have the meaning given for R₁ and R₂ in formula (Ia)

As examples of compounds of formula (Ig), there may be mentioned oxazolidine-2-one, benzamide, acetamide.

The invention also applies to sulfonamide-type compounds.

They may correspond to the following formula:

R₁₇—SO₂—NH—R₁₈ (Ih)

in said formula (Ih), R₁₇ and R₁₈ have the meaning given for R₁ and R₂ in formula (Ia)

As examples of compounds of formula (Ih), tosylhydrazide may be mentioned.

As other types of nucleophilic substrates, there may be mentioned the urea derivative such as the guanidines and which may be represented by formula (II):

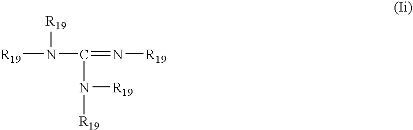

in said formula (II), the groups R₁₉, which are identical or different have the meaning given for R₁ and R₂ in formula (Ia).

As examples of compounds of formula (II), N,N,N′,N′-tetramethylguanidine may be mentioned.

Another category of nucleophiles which may be used in the method of the invention are the amino acids and their derivatives:

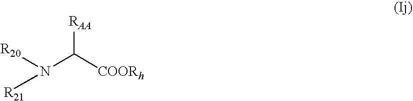

in this formula:
R_{AA} represents the residue of an amino acid, preferably a hydrogen atom, a linear or branched C₁ to C₁₂ alkyl group optionally carrying a functional group, a C₆ to C₁₂ aryl or arylalkyl group or a functional group, preferably a hydroxyl group,
R₂₀ and R₂₁ have the meaning given for R₁ and R₂ in formula (Ia),
Rₕ represents a hydrogen atom, a metal cation, preferably an alkali metal cation or a hydrocarbon group having from 1 to 12 carbon atoms, preferably a C₁ to C₁₂ alkyl group.

In formula (Ij), R_{AA} represents an alkyl group capable of carrying a functional group and there may be mentioned, inter alia, an —OH, —NH₂, —CO—NH₂, —NH—CNH—, —HN—C(O)—NH₂—COOH, —SH or —S—CH₃ group, or an imidazole, pyrrole or pyrazole group.

There may be mentioned, as examples of amino acids, glycine, cysteine, aspartic acid, glutamic acid and histidine.

Nucleophilic substrates which are completely suitable for carrying out the method of the invention are heterocyclic derivatives comprising at least one nucleophilic atom such as a nitrogen, sulfur or phosphorus atom.

More specifically, they correspond to general formula (Ik):

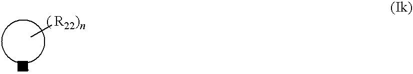

in said formula (Ik):
A symbolizes the residue of a ring forming all or part of a monocyclic or polycyclic, aromatic or nonaromatic heterocyclic system in which one of the carbon atoms is replaced by at least one nucleophilic atom such as a nitrogen, sulfur or phosphorus atom,
R₂₂, which are identical or different, represent substituents on the ring,
n represents the number of substituents on the ring.

The invention applies in particular to the monocyclic heterocyclic compounds corresponding to formula (Ik) in which A symbolizes a saturated or unsaturated, or aromatic heterocycle containing in particular 5 or 6 atoms in the ring which may comprise from 1 to 3 heteroatoms, preferably nitrogen, sulfur and oxygen atoms, and in which at least one of them is a nucleophilic atom such as NH or S.

A may also represent a polycyclic heterocyclic compound defined as consisting of at least 2 aromatic or non-aromatic heterocycles containing at least one heteroatom in each ring and forming with each other ortho- or ortho- and peri-fused systems or a group consisting of at least one aromatic or nonaromatic carbocycle and at least one aromatic or nonaromatic heterocycle forming with each other ortho- or ortho- and peri-fused systems.

It is also possible to start with a substrate resulting from the linkage of a saturated, unsaturated or aromatic heterocycle as mentioned above and a saturated, unsaturated or aromatic carbocycle. The expression carbocycle is preferably understood to mean a ring of the cycloaliphatic or aromatic type having from 3 to 8 carbon atoms, preferably 6.

It should be noted that the carbon atoms of the heterocycle may be optionally substituted, in their entirety or for some of them only with R₂₂ groups.

The number of substituents present on the ring depends on the number of atoms in the ring and on the presence or absence of unsaturations in the ring.

The maximum number of substituents which may be carried by a ring is easily determined by persons skilled in the art.

In formula (Ik), n is a number less than or equal to 4, preferably equal to 0 or 1.

Examples of substituents are given below but this list does not imply any limitation.

The group(s) R₂₂, which are identical or different, preferably represent one of the following groups:
a linear or branched alkyl group of C₁ to C₆, preferably of C₁ to C₄ carbon atoms, such as methyl, ethyl, propyl, isopropyl, butyl, isobutyl, sec-butyl, tert-butyl,
a linear or branched, C₂ to C₆, preferably C₂ to C₄, alkenyl or alkynyl group, such as vinyl, allyl,
a linear or branched C₁ to C₆, preferably C₁ to C₄, alkoxy or thioether group such as the methoxy, ethoxy, propoxy, isopropoxy and butoxy groups, an alkenyloxy group, preferably an allyloxy group or a phenoxy group,
a cyclohexyl, phenyl or benzyl group,
a group or functional group such as: hydroxyl, thiol, carboxylic, ester, amide, formyl, acyl, aroyl, amide, urea, isocyanate, thioisocyanate, nitrile, azide, nitro, sulfone, sulfonic, halogen, pseudohalogen, trifluoromethyl.

The present invention applies most particularly to the compounds corresponding to formula (Ik) in which the group(s) R₂₂ represent more particularly an alkyl or alkoxy group.

More particularly, the optionally substituted residue A represents one of the following rings:
a monocyclic heterocycle comprising one or more heteroatoms:

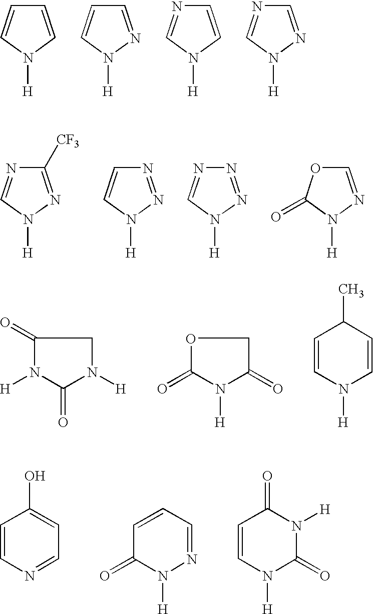

a bicycle comprising a carbocycle and a heterocycle comprising one or more heteroatoms:

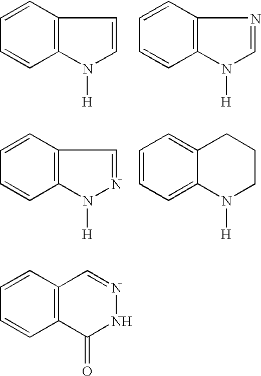

a tricycle comprising at least one carbocycle or one heterocycle comprising one or more heteroatoms:

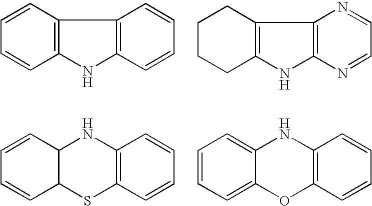

As examples of heterocyclic compounds, preference is given to the use of those which correspond to formula (Ik) in which A represents a ring such as: imidazole, pyrazole, triazole, pyrazine, oxadiazole, oxazole, tetrazole, indole, pyrrole, phthalazine, pyridazine, oxazolidine.

As regards the nucleophilic compounds which may also be used in the method of the invention, there may also be mentioned the compounds of alcohol type or of thiol type which may be represented by the following formula:

R₂₃-Z (Im)

in said formula (Im):
R₂₃ represents a hydrocarbon group having from 1 to 20 atoms and has the meaning given for R₁ or R₂ in formula (Ia),
Z represents a group of the OM₁ or SM₁ type in which M₁ represents a hydrogen atom or a metal cation, preferably an alkali metal cation.

The preferred compounds correspond to formula (Im) in which R₂₃ represents a hydrocarbon group having from 1 to 20 carbon atoms which may be a saturated or unsaturated, linear or branched acyclic aliphatic group; a monocyclic or polycyclic, saturated, unsaturated or aromatic carbocyclic or heterocyclic group; a succession of the abovementioned groups.

More specifically, R₂₃ preferably represents a saturated, linear or branched acyclic aliphatic group preferably having from 1 to 12 carbon atoms, and more preferably still from 1 to 4 carbon atoms.

The invention does not exclude the presence of an unsaturation on the hydrocarbon chain such as one or more double bonds which may be conjugated or nonconjugated, or a triple bond.

As mentioned for R₁ defined in formula (Ia), the hydrocarbon chain may be optionally interrupted by a heteroatom, a functional group or a group carrying one or more substituents.

In formula (Im), R₂₃ may also represent a saturated or unsaturated carbocyclic group preferably having 5 or 6 carbon atoms in the ring; a saturated or unsaturated heterocyclic group containing in particular 5 or 6 atoms in the ring including 1 or 2 heteroatoms such as nitrogen, sulfur, oxygen or phosphorus atoms; a monocyclic, aromatic carbocyclic or heterocyclic group, preferably phenyl, pyridyl, furyl, pyranyl, thiofenyl, thienyl, phospholyl, pyrazolyl, imidazolyl, pyrrolyl or a fused or nonfused polycyclic, aromatic carbocyclic or heterocyclic group, preferably naphthyl.

Since R₂₃ comprises a ring, the latter may also be substituted. The nature of the substituent may be any insofar as it does not interfere with the main reaction. The number of substituents is generally at most 4 per ring but is most often equal to 1 or 2. Reference may be made to the definition of R₂₂ in formula (Ik).

The invention also relates to the case where R₂₃ comprises a succession of aliphatic and/or cyclic, carbocyclic and/or heterocyclic groups.

An acyclic aliphatic group may be attached to a ring by a valency bond, a heteroatom or a functional group such as oxy, carbonyl, carboxyl, sulfonyl and the like.

Reference may be made more particularly to the cycloalkylalkyl, for example cyclohexylalkyl, groups or the aralkyl groups having from 7 to 12 carbon atoms, in particular benzyl or phenylethyl groups.

The invention also envisages a succession of carbocyclic and/or heterocyclic groups, and more particularly a succession of phenyl groups separated by a valency bond or a functional atom or group such as: oxygen, sulfur, sulfo, sulfonyl, carbonyl, carbonyloxy, imino, carbonylimino, hydrazo, C₁-C₁₀, preferably C₁, alkylenediimino.

The saturated or unsaturated, linear or branched acyclic aliphatic group may optionally carry a cyclic substituent. The expression ring is understood to mean a saturated, unsaturated or aromatic carbocyclic or heterocyclic ring.

The preferred compounds of formula (Im) correspond more particularly to general formula (Im₁):

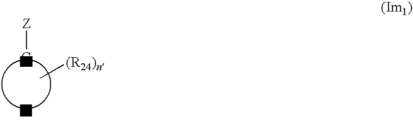

in which:
B symbolizes the residue of a monocyclic or polycyclic aromatic carbocyclic group or a divalent group consisting of a succession of two or more monocyclic aromatic carbocyclic groups,
R₂₄ represents one or more substituents which are identical or different,
Z represents a group of the OM₁ or SM₁ type in which M₁ represents a hydrogen atom or a metal cation, preferably an alkali metal cation,
n′ is a number less than or equal to 5.

As examples of substituents R₂₄, reference may be made to those of formula R₂₂ defined in formula (Ik).

Among the compounds of formula (Im₁), use is made more particularly of those in which the residue (B) represents:
a monocyclic or polycyclic aromatic carbocyclic group with rings which can form with each other an ortho-fused system corresponding to formula (F₁₁):

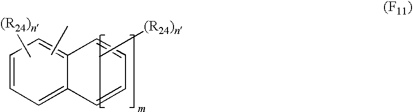

in said formula (F₁₁), m represents a number equal to 0, 1 or 2 and the symbols R₂₄ and n′, which are identical or different, having the meaning given above,
a group consisting of a succession of two or more monocyclic aromatic carbocyclic groups corresponding to formula (F₁₂):

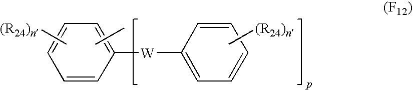

in said formula (F₁₂), the symbols R₂₄ and n′, which are identical or different, have the meaning given above, p is a number equal to 0, 1, 2 or 3 and w represents a valency bond, an alkylene or alkylidene group of preferably C₁ to C₄, a methylene or isopropylidene group or a functional group such as G.

The compounds of formula (Im) preferably used correspond to the formulae (F₁₁) and (F₁₂) in which:
R₂₄ represents a hydrogen atom, a hydroxyl group, a —CHO group, a —NO₂ group, a linear or branched alkyl or alkoxy group having from 1 to 6 carbon atoms, preferably from 1 to 4 carbon atoms, and more preferably methyl, ethyl, methoxy or ethoxy,
w symbolizes a valency bond, an alkylene or alkylidene group having from 1 to 4 carbon atoms or an oxygen atom,
m is equal to 0 or 1,
n′ is equal to 0, 1 or 2,
p is equal to 0 or 1.

By way of illustration of compounds corresponding to formula (Im), there may be mentioned more particularly:
those in which the residue B corresponds to formula (F₁₁) in which m and n′ are equal to 0, such as phenol, thiophenol,
those in which the residue B corresponds to formula (F₁₁) in which m is equal to 0 and n′ is equal to 1, such as hydroquinone, pyrocatechol, resorcinol, alkylphenols, alkylthiophenols, alkoxyphenols, salicylic aldehyde, p-hydroxybenzaldehyde, methyl salicylate, methyl ester of p-hydroxybenzoic acid, chlorophenols, nitrophenols, p-acetamidophenol,
those in which the residue B corresponds to formula (F₁₁) in which m is equal to 0 and n′ is equal to 2, such as dialkylphenols, vanillin, isovanillin, 2-hydroxy-5-acetamidobenzaldehyde, 2-hydroxy-5-propionamidobenzaldehyde, 4-allyloxybenzaldehyde, dichlorophenols, methylhydroquinone, chlorohydroquinone,
those in which the residue B corresponds to formula (F₁₁) in which m is equal to 0 and n′ is equal to 3, such as 4-bromovanillin, 4-hydroxyvanillin, trialkylphenols, 2,4,6-trinitrophenol, 2,6-dichloro-4-nitrophenol, trichlorophenols, dichlorohydroquinones, 3,5-dimethoxy-4-hydroxybenzaldehyde,
those in which the residue B corresponds to formula (F₁₁) in which m is equal to 1 and n′ is equal to 1, such as dihydroxynaphthalene, 4-methoxy-1-naphthol, 6-bromo-2-naphthol,
those in which the residue B corresponds to formula (F₁₂) in which p is equal to 1 and n′ is greater than or equal to 1, such as 2-phenoxyphenol, 3-phenoxyphenol, phenylhydroquinone, 4,4′-dihydroxybiphenyl, 4,4′-isopropylidenediphenol (bisphenol-A), bis(4-hydroxyphenyl)methane, bis(4-hydroxyphenyl)sulfone, bis(4-hydroxyphenyl)sulfoxide, tetrabromobisphenol A.

As nucleophilic compounds of any other nature, there may also be mentioned the phosphorus-containing or phosphorus- and nitrogen-containing compounds, and more particularly those corresponding to the following formulae:
the phosphides of formula (R₂₅)₂—P⁻ (In)
the phosphines of formula (R₂₅)₃—P (Io)
the phosphonium azayldiides of formula (R₂₅)₃—P⁺—N²⁻ (Ip)
the phosphonium azaylides of formula (R₂₅)₃—P⁺—N⁻—R₂₆ (Iq)
in the formulae (In) to (Iq), the groups R₂₅, which are identical or different, and the group R₂₆ represent:
   an alkyl group having from 1 to 12 carbon atoms,
   a cycloalkyl group having 5 or 6 carbon atoms,
   a cycloalkyl group having 5 or 6 carbon atoms, substituted with one or more alkyl radicals having 1 to 4 carbon atoms, or alkoxy radicals having 1 or 4 carbon atoms,
   a phenylalkyl group in which the aliphatic part contains from 1 to 6 carbon atoms,
   a phenyl group,
   a phenyl group substituted with one or more alkyl radicals having 1 to 4 carbon atoms or alkoxy radicals having from 1 to 4 carbon atoms or with one or more halogen atoms.

As more particular examples of phosphorus-containing compounds, there may be mentioned in particular tricyclohexylphosphine, trimethylphosphine, triethylphosphine, tri-n-butylphosphine, triisobutylphosphine, tri-tert-butylphosphine, tribenzylphosphine, dicyclohexylphenylphosphine, triphenylphosphine, dimethylphenylphosphine, diethylphenylphosphine, di-tert-butyl-phenylphosphine.

Other nucleophilic compounds which may be used in the method of the invention are hydrocarbon derivatives comprising a nucleophilic carbon.

There may be mentioned more particularly malonate-type anions comprising an —OOC—HC⁻—COO— group.

There may be mentioned the alkyl malonate anions corresponding respectively to the formulae (Ir):

R₂₇—OOC—C⁻(R₂₈)—COO—R₂₇′ (Ir₁)

R₂₇—OOC—C⁻(R₂₈)—CN (Ir₂)

in said formulae (Ir₁) and (Ir₂)
R₂₇ and R₂₇′, which are identical or different, represent an alkyl group having from 1 to 12 atoms in the alkyl group, preferably from 1 to 4 atoms,
R₂₈ represents:
   a hydrogen atom,
   an alkyl group having from 1 to 12 carbon atoms,
   a cycloalkyl group having 5 or 6 carbon atoms,
   a cycloalkyl group having 5 or 6 carbon atoms, substituted with one or more alkyl radicals having 1 to 4 carbon atoms or alkoxy radicals having 1 or 4 carbon atoms,
   a phenyl group,
   a phenyl group substituted with one or more alkyl radicals having 1 to 4 carbon atoms or alkoxy radicals having from 1 to 4 carbon atoms or with one or more halogen atoms,
   a phenylalkyl group in which the aliphatic part contains from 1 to 6 carbon atoms.

As examples, there may be mentioned in particular dimethyl or diethyl malonate.

Mention may also be made of the malodinitrile-type anions comprising a group NC—C⁻(R₂₈)—CN in which R₂₈ has the definition given above.

Also suitable as nucleophilic compounds are the compounds comprising an anion CN⁻ such as for example sodium or potassium cyanide or a generator of said anion such as, for example, ethanal cyanohydrin CH₃CH(OH)CN or CH₃C(CH₃) (OH)CN.

Also capable of being used in the method of the invention are the acetylenide-type compounds.

They may be schematically represented by formula (Is):

R₂₉—C≡⁻ (Is)

in said formula, R₂₉ is of any nature and the counter-ion is a metal cation, preferably a sodium or potassium atom.

For the meaning of R₂₉, reference may be made to the meanings of R₁.

As more particular examples, there may be mentioned sodium or potassium acetylide or diacetylide.

As other classes of nucleophilic compounds which may be used in the method of the invention, there may be mentioned the profen-type compounds and derivatives which can be represented by the following formula:

R₃₀—HC⁻—COO—R₃₁ (It)

in said formula:
R₃₀ has the meaning given for R₁,
R₃₁ represents an alkyl group having from 1 to 12 atoms in the alkyl group, preferably from 1 to 4 atoms.

The preferred compounds are those which correspond to formula (It) in which R₃₀ represents an alkyl group having from 1 to 12 carbon atoms, a cycloalkyl group having 5 or 6 carbon atoms and an aryl group having 6 or 12 carbon atoms, or a nitrogen-containing heterocycle having 5 or 6 atoms.

There may also be mentioned, as nucleophilic compounds, those comprising a carbanion and in which the counter-ion is a metal and corresponding to the following formulae:

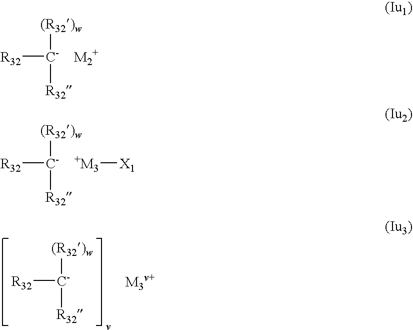

in which:
the group R₃₂ represents:
   an alkyl group having from 1 to 12 carbon atoms,
   a cycloalkyl group having 5 or 6 carbon atoms,
   a cycloalkyl group having 5 or 6 carbon atoms, which is substituted with one or more alkyl radicals having 1 to 4 carbon atoms or alkoxy radicals having 1 or 4 carbon atoms,
   a phenylalkyl group in which the aliphatic part contains from 1 to 6 carbon atoms,
   a phenyl group,
   a phenyl group substituted with one or more alkyl radicals having 1 to 4 carbon atoms or alkoxy radicals having from 1 to 4 carbon atoms or with one or more halogen atoms,
   a saturated, unsaturated or aromatic heterocyclic group preferably comprising 5 or 6 atoms and comprising, as heteroatom, sulfur, oxygen or nitrogen,
the groups R₃₂′ and R₃₂″ represent a hydrogen atom or a group such as R₃₂,
two of the groups R₃₂, R₃₂′ and R₃₂″ may be linked together to form a saturated, unsaturated or aromatic heterocycle preferably having 5 or 6 carbon atoms,
M₂ represents a metal element in group (IA) of the Periodic Table of Elements,
M₃ represents a metal element in groups (IIA), (IIB) of the Periodic Table of Elements,
X₁ represents a chlorine or bromine atom,
v is the valency of the metal M₃,
w is equal to 0 or 1.

In the present text, reference is made below to the Periodic Table of Elements published in the Bulletin de la Société Chimique de France, No. 1 (1966).

Among the compounds of formula (Iu₁) to (Iu₃), those which are preferred involve, as metals, lithium, sodium, magnesium or zinc, and X₁ represents a chlorine atom.

The groups R₃₂, R₃₂′ and R₃₂″ are advantageously a C₁-C₄ alkyl group, a cyclohexyl or phenyl group; or said groups may form a benzene, cyclopentadiene, pyridine or thiofene ring.

As examples, there may be mentioned n-butyllithium, t-butyllithium, phenyllithium, bromide or chloride of methyl- or ethyl- or phenylmagnesium, diphenylmagnesium, dimethyl- or diethylzinc, cyclopentadienezinc, chloride or bromide of ethylzinc.

In the present text, lists of nucleophilic compounds are given which do not imply any limitation and any type of nucleophilic compound may be envisaged.

The preferred nucleophiles used in the method of the invention are the following: diphenylamine, N-methyl-N-phenylamine, benzophenone imine, benzophenone hydrazone, benzophenone oxime.

### Aromatic Compound

In accordance with the method of the invention, a —C≡C— or —C—HE—(O, P, N) bond is created by reacting a nucleophilic compound with a compound comprising an unsaturation at the α-position of a leaving group.

More specifically, this involves a compound comprising a leaving group Y symbolized by the formula (II):

R₀—Y (II)

in said formula (II):
R₀ represents a hydrocarbon group comprising from 2 to 20 carbon atoms and possesses a double bond at the α-position with respect to a leaving group Y or a monocyclic or polycyclic aromatic carbocyclic and/or heterocyclic group carrying a leaving group Y on a ring.

In accordance with the method of the invention, the compound of formula (I) is reacted with a compound of formula (II) in which:
R₀ represents an aliphatic hydrocarbon group comprising a double bond at the α-position with respect to the leaving group or an unsaturated cyclic hydrocarbon group in which an unsaturation carries the leaving group,
R₀ represents a monocyclic or polycyclic aromatic carbocyclic and/or heterocyclic group,
Y represents a leaving group, preferably a halogen atom or a sulfonic ester group of formula —OSO₂—Rₑ, in which R^{e} is a hydrocarbon group.

The compound of formula (II) will be designated, in the text which follows, by “compound carrying a leaving group”.

In the formula for the sulfonic ester group, Rₑ is a hydrocarbon group of any nature. However, given that Y is a leaving group, it is advantageous, from an economic point of view, for Rₑ to be of a simple nature, and represent more particularly a linear or branched alkyl group having from 1 to 4 carbon atoms, preferably a methyl or ethyl group, but it may also represent, for example, a phenyl or tolyl group or a trifluoromethyl group. Among the groups Y, the preferred group is a triflate group, which corresponds to a group Rₑ representing a trifluoromethyl group.

As preferred leaving groups, a bromine atom is preferably chosen.

The compounds of formula (II) most particularly desired according to the method of the invention may be classified into three groups:
(1) those of the aliphatic type carrying a double bond which may be represented by the formula (IIa):

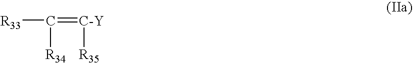

in said formula (IIa):
R₃₃, R₃₄ and R₃₅, which are identical or different, represent a hydrogen atom or a hydrocarbon group having from 1 to 20 carbon atoms, which may be a saturated or unsaturated, linear or branched aliphatic group; a monocyclic or polycyclic, saturated, unsaturated or aromatic carbocyclic or heterocyclic group; a succession of aliphatic and/or carbocyclic and/or heterocyclic groups as mentioned above,
Y symbolizes the leaving group as defined above,
(2) those of the aromatic type which are designated, in the text which follows, by “haloaromatic compound” and which may be represented by the formula (IIb):

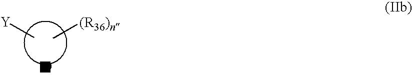

in which:
D symbolizes the residue of a ring forming all or part of a monocyclic or polycyclic aromatic carbocyclic and/or heterocyclic system,
R₃₆, which are identical or different, represent substituents on the ring,
Y represents a leaving group as defined above,
n″ represents the number of substituents on the ring.

The invention applies to the unsaturated compounds corresponding to the formulae (IIa) in which R₃₃ preferably represents a saturated, linear or branched acyclic aliphatic group preferably having from 1 to 12 carbon atoms.

The invention does not exclude the presence of another unsaturation in the hydrocarbon chain such as another triple bond or one or more double bonds which may be conjugated or unconjugated.

The hydrocarbon chain may be optionally interrupted by a heteroatom (for example oxygen or sulfur) or by a functional group insofar as the latter does not react, and there may be mentioned in particular a group such as in particular —CO—.

The hydrocarbon chain may optionally carry one or more substituents insofar as they do not react under the reaction conditions, and there may be mentioned in particular a halogen atom, a nitrile group or a trifluoromethyl group.

The saturated or unsaturated, linear or branched acyclic aliphatic group may optionally carry a cyclic substituent. The expression ring is understood to mean a monocyclic or polycyclic, saturated, unsaturated or aromatic carbocyclic or heterocyclic ring.

The acyclic aliphatic group may be attached to the ring by a valency bond, a heteroatom or a functional group such as oxy, carbonyl, carboxyl, sulfonyl and the like.

As examples of cyclic substituents, it is possible to envisage cycloaliphatic, aromatic or heterocyclic substituents, in particular cycloaliphatic substituents comprising 6 carbon atoms in the ring or benzene substituents, these cyclic substituents themselves optionally carrying any substituent insofar as they do not hamper the reactions which occur in the method of the invention. There may be mentioned in particular alkyl or alkoxy groups having from 1 to 4 carbon atoms.

Among the aliphatic groups carrying a cyclic substituent, reference may be made more particularly to the aralkyl groups having from 7 to 12 carbon atoms, in particular benzyl or phenylethyl.

In formula (IIa), a saturated or unsaturated carbocyclic group preferably having 5 or 6 carbon atoms in the ring, preferably cyclohexyl; a saturated or unsaturated heterocyclic group containing in particular 5 or 6 atoms in the ring including 1 or 2 heteroatoms such as nitrogen, sulfur and oxygen atoms; a monocyclic aromatic carbocyclic group, preferably phenyl, or a fused or unfused polycyclic aromatic carbocyclic group, preferably naphthyl, may also be represented.

As for R₃₄ and R₃₅, they preferably represent a hydrogen atom or an alkyl group having from 1 to 12 carbon atoms, a phenyl group or an aralkyl group having from 7 to 12 carbon atoms, preferably a benzyl group.

In formula (IIa), R₃₃, R₃₄ and R₃₅ represent more particularly a hydrogen atom or R₃₃ represents a phenyl group, and R₃₄ and R₃₅ represent a hydrogen atom.

It should also be noted that the groups R₃₃ and R₃₄ may also represent a functional group insofar as the latter does not interfere with the coupling reaction. Functional groups such as amido, ester, ether and cyano may be mentioned as examples.

As examples of compounds corresponding to formulae (IIa), there may be mentioned in particular vinyl chloride or bromide or β-bromo- or β-chlorostyrene.

The invention applies in particular to the haloaromatic compounds corresponding to formula (IIb) in which D is the residue of a cyclic compound preferably having at least 4 atoms in the ring, preferably 5 or 6, which is optionally substituted, and representing at least one of the following rings:
a monocyclic or polycyclic aromatic carbocycle, that is to say a compound consisting of at least 2 aromatic carbocycles and forming with each other ortho- or ortho- and peri-fused systems or a compound consisting of at least 2 carbocycles only one of which is aromatic and forming with each other ortho- or ortho- and peri-fused systems,
a monocyclic aromatic heterocycle containing at least one of the heteroatoms P, O, N and S or a polycyclic aromatic heterocycle, that is to say a compound consisting of at least 2 heterocycles containing at least one heteroatom in each ring in which at least one of the two rings is aromatic and forming with each other ortho- or ortho- and peri-fused systems or a compound consisting of at least one carbocycle and at least one heterocycle in which at least one of the rings is aromatic and forming with each other ortho- or ortho- and peri-fused systems.

More particularly, the optionally substituted residue D preferably represents the residue of an aromatic carbocycle such as benzene, of an aromatic bicycle comprising two aromatic carbocycles such as naphthalene; a partially aromatic bicycle comprising two carbocycles one of which is aromatic, such as 1,2,3,4-tetrahydronaphthalene.

The invention also envisages the fact that D may represent the residue of a heterocycle insofar as it is more electrophilic than the compound corresponding to formula (Ik).

As specific examples, there may be mentioned an aromatic heterocycle such as furan, pyridine; an aromatic bicycle comprising an aromatic carbocycle and an aromatic heterocycle such as benzofuran, benzopyridine, a partially aromatic bicycle comprising an aromatic carbocycle and a heterocycle such as methylenedioxybenzene; an aromatic bicycle comprising two aromatic heterocycles such as 1,8-naphthypyridine; a partially aromatic bicycle comprising a carbocycle and an aromatic heterocycle such as 5,6,7,8-tetrahydroquinoline.

In the method of the invention, a haloaromatic compound of formula (IIb) is preferably used in which D represents an aromatic nucleus, preferably a benzene or naphthalene nucleus.

The aromatic compound of formula (IIb) may carry one or more substituents.

In the present text, the expression “more” is generally understood to mean less than 4 substituents R₃₆ on an aromatic nucleus.

For examples of substituents, reference may be made to the meaning given for R₂₂ in formula (Ik).

R₃₆ also represents a saturated, unsaturated or aromatic heterocycle comprising 5 or 6 atoms and comprising, as heteroatom, sulfur, oxygen or nitrogen. The pyrazolyl or imidazolyl groups may be mentioned in particular.

In formula (IIb), n″ is a number less than or equal to 4, preferably equal to 1 or 2.

As examples of compounds corresponding to formula (IIb), there may be mentioned in particular bromobenzene, iodobenzene, p-chlorotoluene, p-bromoanisole, p-bromotrifluorobenzene.

The quantity of the unsaturated compound carrying a leaving group of formula (II), preferably of formula (IIa) or (IIb) or used is generally expressed relative to the quantity of the nucleophilic compound close to stoichiometry. Thus, the ratio between the number of moles of the unsaturated compound carrying the leaving group and the number of moles of the nucleophilic compound varies most often between 0.1 and 2.0, preferably between 0.5 and 1.5.

### Catalyst

In accordance with the method of the invention, the nucleophilic compound preferably corresponding to formulae (Ia) to (Iu₃) is reacted with a compound carrying a leaving group corresponding to formula (II), preferably of formula (IIa) or (IIb) in the presence of an effective quantity of a copper-based catalyst and a base.

As examples of catalysts capable of being used, there may be mentioned metallic copper or the organic or inorganic compounds of copper(I) or copper (II).

The catalysts used in the method of the invention are known products.

By way of examples of catalysts of the invention, there may be mentioned in particular, as copper compounds, copper(I) bromide, copper(II) bromide, copper(I) iodide, copper(I) chloride, copper(II) chloride, basic copper(II) carbonate, copper(I) nitrate, copper(II) nitrate, copper(I) sulfate, copper(II) sulfate, copper(I) sulfite, copper(I) oxide, copper(II) oxide, copper(I) acetate, copper(II) acetate, copper(II) trifluoromethylsulfonate, copper(II) hydroxide, copper(I) methoxide, copper(II) methoxide, chlorocopper(II) methoxide of formula ClCuOCH₃.

Copper(I) iodide is preferably chosen.

The quantity of catalyst used, expressed by the molar ratio between the number of moles of copper catalyst expressed as copper and the number of moles of compound carrying the leaving group generally varies between 0.001 and 0.2, preferably between 0.01 and 0.1.

### Base

Also involved in the method of the invention is a base whose role is to trap the leaving group.

The characteristic of the base is that it has a pKa at least greater than or equal to 4, preferably between 6 and 30.

The pKa is defined as the ionic dissociation constant of the acid/base pair when water is used as solvent.

For the choice of a base having a pKa as defined by the invention, reference may be made, inter alia, to Handbook of Chemistry and Physics, 66th edition, p. D-161 and D-162.

Among the bases which can be used, there may be mentioned, inter alia, the inorganic bases such as the carbonates, hydrogen carbonates, phosphates or hydroxides of alkali metals, preferably of sodium, potassium or cesium, or of alkaline-earth metals, preferably calcium, barium or magnesium.

It is also possible to use the alkali metal hydrides, preferably sodium hydride, or the alcoholates of alkali metals, preferably of sodium or potassium, and more preferably sodium methoxide, ethoxide or tert-butoxide.

Also suitable are organic bases such as the tertiary amines and there may be mentioned more particularly triethylamine, tri-n-propylamine, tri-n-butylamine, methyldibutylamine, methyldicyclohexylamine, ethyldiisopropylamine, N,N-diethylcyclohexylamine, pyridine, 4-dimethylaminopyridine, N-methylpiperidine, N-ethylpiperidine, N-n-butylpiperidine, 1,2-dimethylpiperidine, N-methylpyrrolidine, 1,2-dimethylpyrrolidine.

Among the bases, the alkali metal carbonates are preferably chosen.

The quantity of base used is such that the ratio between the number of moles of base and the number of moles of the aromatic compound carrying the leaving group preferably varies between 1 and 4, preferably in the region of 2.

### Nitrile-Type Solvent

In accordance with the method of the invention, the arylation or vinylation reaction carried out according to the invention is carried out in a nitrile-type solvent which may be symbolized by the following formula:

Rₕ—CN (III)

in said formula (III):
Rₕ represents a hydrocarbon group comprising at least one nitrile group, having from 1 to 24 carbon atoms, optionally substituted, which may be a saturated or unsaturated, linear or branched acyclic aliphatic group; a monocyclic or polycyclic, saturated, unsaturated or aromatic cycloaliphatic group; a saturated or unsaturated, linear or branched aliphatic group, carrying a cyclic substituent.

The solvent preferably chosen corresponds to formula (III) in which Rₕ represents a saturated or unsaturated, linear or branched acyclic aliphatic group.

Rₕ preferably represents a saturated, linear or branched acyclic aliphatic group preferably having from 1 to 12 carbon atoms.

The invention does not exclude the presence of an unsaturation in the hydrocarbon chain such as a double or triple bond or one or more double bonds which may be conjugated or unconjugated.

The hydrocarbon chain may be optionally interrupted by a heteroatom (for example oxygen or sulfur) or by a functional group insofar as it does not react and there may be mentioned in particular a group such as especially —CO—.

The hydrocarbon chain may optionally carry one of the following substituents: —ORᵢ, —NRᵢRᵢ in these formulae, the groups Rᵢ, which are identical or different, represent hydrogen or a linear or branched alkyl group having from 1 to 4 carbon atoms, preferably a methyl or ethyl group or a phenyl group.

The saturated or unsaturated, linear or branched acyclic aliphatic group may optionally carry a cyclic substituent. The expression ring is understood to mean a saturated, unsaturated or aromatic carbocyclic or heterocyclic ring.

The acyclic aliphatic group may be attached to the ring by a valency bond, a heteroatom or a functional group such as oxy, carbonyl, carboxyl, sulfonyl and the like.

The saturated or unsaturated, linear or branched acyclic aliphatic residue may optionally carry a cyclic substituent. The expression ring is understood to mean a saturated, unsaturated or aromatic carbocyclic or heterocyclic ring.

The acyclic aliphatic residue may be attached to the ring by a valency bond or by an atom or a functional group, for example —O—.

As examples of cyclic substituents, it is possible to envisage cycloaliphatic, aromatic or heterocyclic substituents, in particular cycloaliphatic substituents comprising 6 carbon atoms in the ring, or benzene or heterocyclic substituents comprising 5 or 6 atoms in the ring including one or two heteroatoms such as nitrogen (not substituted with a hydrogen atom), sulfur and oxygen atoms. It is possible for there to be a substituent insofar as they do not interfere with the coupling reaction. There may be mentioned in particular the alkyl or alkoxy groups having from 1 to 4 carbon atoms.

In general formula (III), Rₕ may also represent a saturated carbocyclic group or a group comprising 1 or 2 unsaturations in the ring, generally having from 3 to 7 carbon atoms, preferably 6 carbon atoms in the ring; it being possible for said ring to be substituted with 1 to 5 groups R₅, preferably 1 to 3, R₅ having the meanings set out above.

As preferred examples of groups Rₕ, there may be mentioned the cyclohexyl or cyclohexenyl groups, optionally substituted with linear or branched alkyl groups having from 1 to 4 carbon atoms.

In general formula (III), Rₕ may also represent an aromatic group, preferably a group having 6 carbon atoms.

The solvents used may be of the mono- or polynitrile type. It is also possible to use a mixture of solvents.

As examples of solvents used, aliphatic or aromatic nitriles, preferably acetonitrile, propionitrile, butanenitrile, isobutanenitrile, pentanenitrile, 2-methylglutaronitrile, adiponitrile, benzonitrile, tolunitrile, malonitrile, 1,4-benzonitrile, are used.

The quantity of organic solvent to be used is determined according to the nature of the organic solvent chosen.

It is determined such that the concentration of the compound carrying the leaving group in the organic solvent is preferably between 0.5 and 2 mol/liter of organic solvent.

The arylation or vinylation reaction of the nucleophilic compound takes place at a temperature which is advantageously between 0° C. and 120° C., preferably between 20° C. and 100° C., and more preferably still between 25° C. and 80° C.

The arylation or vinylation reaction is generally used under atmospheric pressure, but it is also possible to use higher pressures which may be up to for example 10 bar.

From a practical point of view, the reaction is simple to carry out.

The order in which the reagents are used is not critical. Preferably, the catalyst, preferably the copper catalyst, the nucleophilic compound of formula (I), the base, the compound carrying the leaving group of formula (II) and the organic solvent are loaded.

The reaction medium is brought to the desired temperature.

The progress of the reaction is checked by monitoring the disappearance of the compound carrying the leaving group.

At the end of the reaction, a product of the R-Nu-R₀ type is obtained, R representing the residue of the nucleophilic compound, and more particularly an arylated product comprising the residue of the nucleophilic compound and the residue of the electrophilic compound which preferably corresponds to the following formula (IV):

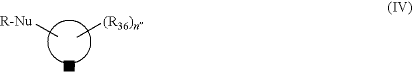

in said formula (IV), D, R, R₃₆ and n″ have the meaning given above and Nu represents an oxygen or nitrogen atom.

The compound obtained is recovered according to the conventional techniques used, in particular by crystallization from an organic solvent.

As more specific examples of organic solvents, there may be mentioned in particular halogenated or nonhalogenated aliphatic or aromatic hydrocarbons, carboxamides, nitrites. There may be mentioned in particular cyclohexane, toluene, dimethylformamide, acetonitrile or dichloromethane.

It is also possible to recover the product on a silica chromatography column.

Exemplary embodiments of the invention are given below. These examples are given as a guide, with no limitation being implied.

Before detailing the examples, an operating protocol is given which is repeated in all the examples, unless otherwise stated.

In the examples, the rate of conversion (RC) corresponds to the ratio between the number of substrate converted and the number of moles of substrate used.

The yield (AY) corresponds to the ratio between the number of moles of product formed and the number of moles of substrate used.

### EXAMPLES

Two different procedures are given depending on the physical form, a solid or a liquid, of the nucleophilic compound and of the arylation agent.

### Operating Protocol A: Case of a Solid Nucleophilic Compound and a Liquid Arylation Agent

Copper(I) oxide (14.4 mg; 0.1 mmol) or copper(I) iodide (19.04 mg; 0.1 mmol), 2 or 3 mmol of nucleophilic compound (1 or 1.5 equivalents) and 1.303 g of cesium carbonate (4 mmol) are successively introduced into a 35 ml Schlenk tube, previously dried in an oven at 100° C., provided with a magnetic stirrer bar (12×4.5 mm) and placed under a nitrogen atmosphere.

The Schlenk tube is purged under vacuum and then filled again with nitrogen. 2 or 3 mmol of arylation agent (1 or 1.5 equivalents) and then 1.2 ml of anhydrous acetonitrile are then added thereto by means of syringes.

The reactor is placed in an oil bath at the temperature of 82° C. and stirred for a period varying from one to five days.

At the end of this period, the reaction mixture is diluted with 25 ml of dichloromethane, filtered on celite, totally concentrated under reduced pressure and then taken up in 50 ml of dichloromethane.

This organic phase is extracted with distilled water (2×20 ml).

The aqueous phase is re-extracted with 20 ml of dichloromethane.

The overall organic phase is washed with a saturated aqueous sodium chloride solution (2×20 ml), dried over MgSO₄, filtered and concentrated under reduced pressure.

The residue obtained is purified by chromatography on a silica column (35-70 μm).

### Operating Protocol B: Case of a Liquid Nucleophilic Compound and a Liquid Arylation Agent

Copper(I) oxide or copper(I) iodide and cesium carbonate are successively introduced into a 35 ml Schlenk tube, previously dried in an oven at 100° C., provided with a magnetic stirrer bar (12×4.5 mm) and placed under a nitrogen atmosphere.

The Schlenk tube is purged under vacuum and then filled again with nitrogen.

The nucleophilic compound, the arylation agent and 1.2 ml of anhydrous acetonitrile are then added thereto by means of syringes.

The subsequent steps are rigorously identical to those of the general procedure A.

### Example 1

### Arylation of Nitrogen-Containing Nucleophilic Compounds

### Example 1.1

### Synthesis of 1-phenyl-1H-pyrazole

The general procedure A (CuI, 82° C., 24 hours) was followed using 211 μl of bromobenzene (2 mmol), 204 mg of pyrazole (3 mmol) and 1.2 ml of acetonitrile.

The residue obtained was purified by chromatography on a silica column (eluent: hexane/dichloromethane 90/10 to 70/30). Yield: 65% (colorless oil).

The compound obtained corresponds to the following formula:

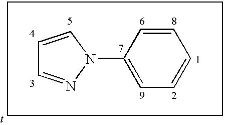

The characteristics are the following:

b.p.: 58° C. under 0.2 mmHg.

¹H NMR/CDCl₃ (250 MHz): δ 7.92 (dd, 1H, ³J_{HH}=2.4 Hz, ⁴J_{HH}=0.5 Hz, H₅), 7.70 (m, 3H, H_{3,6,9}), 7.45 (m, 2H, H_{2,8}), 7.29 (m, 1H, H₁), 6.46 (dd, 1H, ³J_{HH}=2.4 Hz, ³J_{HH}=1.8 Hz, H₄).

¹³C NMR/CDCl₃: δ 141.08 (C₃), 140.23 (C₇), 129.43 (C_{2,8}), 126.75 (C₅), 126.44 (C₁), 119.21 (C₆₋₉), 107.61 (C₄).

GC/MS: tr=13.15 min, M/Z=144, purity=100%.

IR (KBr): ν (cm⁻¹)=3142, 3121 and 3150 (ff, aromatic), 1601, 1521 and 1501 (FF), 1464 (f), 1393 and 1332 (FF), 1253 (f), 1198 and 1120 (F), 1074 (f), 1046 (FF), 1036 (F), 936 (FF), 915 and 905 (f), 756 (FF).

Rf=0.40 (eluent: dichloromethane/petroleum ether 60/40).

### Examples 1.2 to 1.4

In this series of tests, the operating protocol A is repeated under the following conditions: 0.5 mmol of phenyl iodide (1.67 M), 1.5 equivalents of nitrogen-containing nucleophilic compound, 10% of CuI and 2 equivalents of CsCO₃ in the acetonitrile solvent.

The reaction temperature is 82° C. and the duration of the reaction is 24 hours.

The results are presented in the following table:

TABLE I

| Ref. | Haloaromatic | Nucleophilic | Product | |
|---|---|---|---|---|
| ex. | compound | compound | obtained | Yield |
| 1.2 | Ph-I | 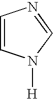 | 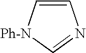 | 57 |
| 1.3 | Ph-I | 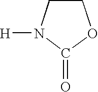 | 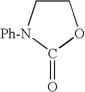 | 41 |
| 1.4 | Ph-I | Ph-SO₂—NH₂ | Ph-SO₂—NH-Ph | 25 |

### Example 2

### Arylation of Oxygenated Nucleophilic Compounds

### Example 2.1

### Synthesis of 3,5-dimethylphenyl and phenyl ether

The general procedure A (Cu₂O, 82° C., 29 hours) was followed using 336 μl of iodobenzene (3 mmol), 244 mg of 3,5-dimethylphenol (2 mmol), 600 mg of ground and activated 3 Å molecular sieve and 1.2 ml of acetonitrile.

The brown oil obtained at the end of the treatment was purified by chromatography on a silica column (eluent: hexane).

The yield obtained is 71% (colorless oil).

The compound obtained corresponds to the following formula:

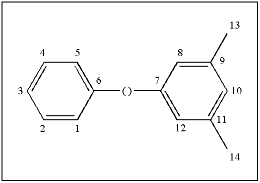

The characteristics are the following:

¹H NMR/CDCl₃: δ 7.28-7.42 (m, 2H, H_{2,4}), 7.12-7.17 (m, 1H, H₃), 7.03-7.14 (m, 2H, H_{1,5}), 6.79 (m, 1H, H₁₀), 6.69 (m, 2H, H_{8,12}), 2.33 (s, 6H, H_{13,14}).

¹³C NMR/CDCl₃: δ 157.50 (C₆), 157.22 (C₇), 139.61 (C_{9,11}), 129.70 (C_{2,4}), 125.04 (C₁₀), 123.02 (C₃), 118.89 (C_{1,5}), 116.67 (C_{8,12}), 21.35 (C₁₃).

GC/MS: tr=16.87 min, M/Z=198, purity=98%.

Rf=0.19 (eluent: hexane).

### Example 3

### Arylation of Nucleophilic Carbon Compounds

### Example 3.1

### Synthesis of diethyl 2-phenylmalonate

The general procedure B (70° C., 24 hours) was followed using 38 mg of copper(I) iodide (0.2 mmol), 224 μl of iodobenzene (2 mmol), 607 μl of diethyl malonate (4 mmol), 600 mg of ground and activated 3 Å molecular sieve, 977 mg of cesium carbonate (3 mmol) and 1.2 ml of acetonitrile.

The reaction mixture is neutralized with 6 ml of a 1 N aqueous hydrochloric acid solution before being filtered on celite.

The filtrate is extracted with dichloromethane and then concentrated under reduced pressure.

The residue obtained was purified directly by chromatography on a silica column (eluent: hexane/dichloromethane 100/0 to 80/20).

The yield obtained is 53% (colorless oil).

The compound obtained corresponds to the following formula:

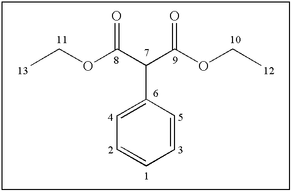

The characteristics are the following:

¹H NMR/CDCl₃: δ 7.32-7.42 (m, 5H, H_{1,5}), 4.62 (s, 1H, H₇), 4.22 (m, 4H, H_{10,11}), 1.26 (t, 6H, ³J_{HH}=7.1 Hz, H_{12,13}). The protons of each methylene fragment of the ester functional group are diastereotopic and lead to the obtaining of an unresolved complex of the second order, which was not analyzed.

¹³C NMR/CDCl₃: δ 168.15 (C_{8,9}), 132.86 (C₆), 129.27 (C_{2,3}), 128.58 (C_{4,5}), 128.18 (C₁), 61.77 (C_{11,12}), 58.00 (C₇), 14.00 (C_{12,13}).

GC/MS: tr=16.77 min, M/Z=236, purity=99%.

Rf=0.27 (eluent: hexane/dichloromethane 70/30).

1-29. (canceled)

30. A method for creating a carbon-carbon or carbon-heteroatom bond by the reaction of an unsaturated compound carrying a leaving group and a nucleophilic substrate or compound donating a carbon atom or a heteroatom (HE) capable of substituting for the leaving group, thus creating a C—C or C—HE bond, in the presence of a copper-based catalyst and a base, the reaction taking place in the absence of a ligand and in a nitrile solvent.

31. The method as claimed in claim 30, wherein the nucleophilic substrate is an organic hydrocarbon compound which is acyclic or cyclic and whose characteristic is to comprise at least one atom carrying a free doublet optionally having a charge, and optionally a nitrogen, oxygen, sulfur, phosphorus or carbon atom.

32. The method as claimed in claim 30, wherein the nucleophilic substrate has at least one atom or group below:

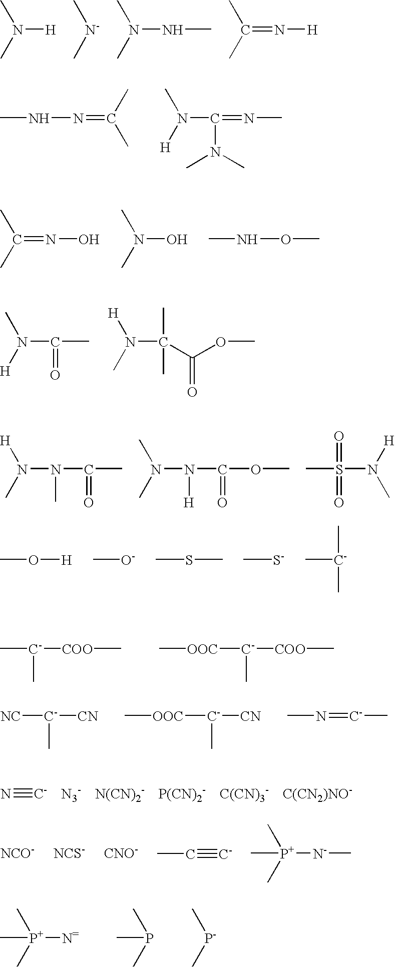

33. The method as claimed in claim 30, wherein the nucleophilic substrate has at least one nitrogen atom carrying a free doublet contained in a saturated, unsaturated or aromatic ring: the ring optionally having from 3 to 8 atoms.

34. The method as claimed in claim 31, wherein the nucleophilic substrate is a primary or secondary amine; an imine; an oxime; a hydroxylamine; a hydrazine; a hydrazone; an amide; a sulfoamide; a urea derivative; an amino acid; or a heterocyclic derivative, optionally containing nitrogen, sulfur or phosphorus.

35. The method as claimed in claim 31, wherein the nucleophilic substrate corresponds to the following formula: 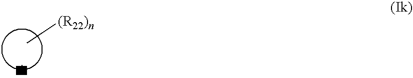
wherein:
A symbolizes the residue of a ring forming all or part of a monocyclic or polycyclic, aromatic or nonaromatic heterocyclic system in which one of the carbon atoms is replaced by at least one nucleophilic optionally a nitrogen, sulfur or phosphorus atom,
R₂₂, which are identical or different, represent substituents on the ring, and n represents the number of substituents on the ring.

36. The method as claimed in claim 35, wherein the nucleophilic substrate corresponds to formula (Ik) in which A represents a imidazole, pyrazole, triazole, pyrazine, oxadiazole, oxazole, tetrazole, indole, pyrrole, phthalazine, pyridazine, or oxazolidine ring.

37. The method as claimed in claim 31, wherein the nucleophilic substrate is an alcohol or thiol compound, optionally a hydroxy or thioaromatic compound.

38. The method as claimed in claim 37, wherein the nucleophilic substrate corresponds to the following formula: 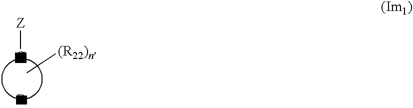
in which:
B symbolizes the residue of a monocyclic or polycyclic aromatic carbocyclic group or a divalent group consisting of a succession of two or more monocyclic aromatic carbocyclic groups,
R₂₄ represents one or more substituents which are identical or different,
Z represents a group of the OM₁ or SM₁ type in which M₁ represents a hydrogen atom or a metal cation, optionally an alkali metal cation, and
n′ is a number less than or equal to 5.

39. The method as claimed in claim 31, wherein the nucleophilic substrate is a hydrocarbon compound having a nucleophilic carbon, optionally a malonate, a cyanomalonate, or a malodinitrile, a compound having a cyanide anion or its generator, an acetylenide, a profen compound, a nucleophilic compound having a carbanion and whose counter-ion is a metal, optionally lithium, sodium, magnesium or zinc.

40. The method as claimed in claim 31, wherein the nucleophilic substrate is a phosphide, a phosphine, a phosphonium azayldiide, or a phosphonium azaylide.

41. The method as claimed in claim 30, wherein the nucleophilic compound is: pyrazole, oxazolidin-2-one, phenylsulfonamide, 3,5-dimethylphenol, dimethyl malonate or diethyl malonate.

42. The method as claimed in claim 30, wherein the compound carrying a leaving group Y is symbolized by the formula (II):
R₀-Y (II)
wherein:
R₀ represents a hydrocarbon group having from 2 to 20 carbon atoms and possesses a double bond at the α-position with respect to a leaving group Y or a monocyclic or polycyclic aromatic carbocyclic and/or heterocyclic group carrying a leaving group Y on a ring.

43. The method as claimed in claim 42, wherein the compound having a leaving group corresponds to formula (II) in which:
R₀ represents an aliphatic hydrocarbon group having a double bond at the α-position with respect to the leaving group or an unsaturated cyclic hydrocarbon group in which an unsaturation carries the leaving group,
R₀ represents a monocyclic or polycyclic aromatic carbocyclic and/or heterocyclic group, and
Y represents a leaving group, optionally a halogen atom or a sulfonic ester group of formula —OSO₂—Rₑ, in which Rₑ is a hydrocarbon group.

44. The method as claimed claim 42, wherein the leaving group corresponds to formula (II) in which Y represents a bromine or chlorine atom or a sulfonic ester of formula —OSO₂—Rₑ, in which Rₑ is a linear or branched alkyl group having from 1 to 4 carbon atoms, optionally a methyl or ethyl group, a phenyl or tolyl group or a trifluoromethyl group.

45. The method as claimed in claim 42, wherein the compound having a leaving group corresponds to formula (II) and is:
(1) a compound carrying a double bond represented by the formula (IIa): 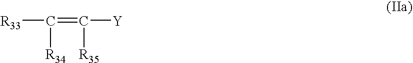
in said formula (IIa):
R₃₃, R₃₄ and R₃₅, which are identical or different, represent a hydrogen atom or a hydrocarbon group having from 1 to 20 carbon atoms, which is a saturated or unsaturated, linear or branched aliphatic group; a monocyclic or polycyclic, saturated, unsaturated or aromatic carbocyclic or heterocyclic group; a succession of aliphatic and/or carbocyclic and/or heterocyclic groups as mentioned above, and
Y symbolizes the leaving group as defined above, or
(2) a haloaromatic compound represented by the formula (IIb) 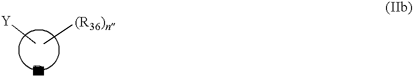
in which:
D symbolizes the residue of a ring forming all or part of a monocyclic or polycyclic aromatic carbocyclic and/or heterocyclic system,
R₃₆, which are identical or different, represent substituents on the ring,
Y represents a leaving group as defined above, and
n″ represents the number of substituents on the ring.

46. The method as claimed in claim 42, wherein the compound carrying a leaving group corresponding to formula (II) is vinyl chloride, vinyl bromide, β-bromostyrene, β-chlorostyrene, bromobenzene, iodobenzene, p-chlorotoluene, p-bromoanisole, or p-bromotrifluorobenzene.

47. The method as claimed in claim 42, wherein the compound carrying a leaving group corresponding to formula (II) is bromobenzene or iodobenzene.

48. The method as claimed in claim 30, wherein the reaction takes place in the presence of a base which is a carbonates, hydrogen carbonate, hydroxide of alkali metals, or of alkaline-earth metal, alkali metal hydrides, alcoholate of alkali metals, methoxide, ethoxide, tert-butoxide; or a tertiary amine.

49. The method as claimed in claim 30, wherein the reaction takes place in the presence of a nitrile solvent of the formula:
Rₕ—CN (III)
in said formula (III):
Rₕ represents a hydrocarbon group comprising at least one nitrile group, having from 1 to 24 carbon atoms, optionally substituted, saturated or unsaturated, linear or branched acyclic aliphatic group; a monocyclic or polycyclic, saturated, unsaturated or aromatic cycloaliphatic group; a saturated or unsaturated, linear or branched aliphatic group, carrying a cyclic substituent.

50. The method as claimed in claim 49, wherein the nitrile solvent is acetonitrile, propionitrile, butanenitrile, isobutanenitrile, pentanenitrile, 2-methylglutaronitrile, adiponitrile, benzonitrile, tolunitrile, malonitrile, or 1,4-benzonitrile.

51. The method as claimed in claim 30, wherein the arylation or vinylation reaction is carried out at a temperature of between 0° C. and 120° C., optionally between 25° C. and 85° C.

52. The method as claimed in claim 30, wherein the copper catalyst is copper(I) bromide, copper(II) bromide, copper(I) iodide, copper(I) chloride, copper(II) chloride, basic copper(II) carbonate, copper nitrate, copper nitrate, copper sulfate, copper sulfate, copper(I) sulfite, copper(I) oxide, copper(II) oxide, copper(I) acetate, copper(II) acetate, copper(II) trifluoromethylsulfonate, copper(II) hydroxide, copper(I) methoxide, copper(II) methoxide, or chlorocopper(II) methoxide of formula ClCuOCH₃.

53. The method as claimed in claim 30, wherein the catalyst is copper(I) iodide.

54. The method as claimed in claim 30, wherein the catalyst is used in a quantity, expressed by the molar ratio between the number of moles of copper catalyst expressed as copper and the number of moles of compound carrying the leaving group, of between 0.001 and 0.2, optionally between 0.01 and 0.1.

55. The method as claimed in claim 30, wherein the nitrile-type solvent is used in a quantity such that the concentration of the compound carrying the leaving group in the organic solvent is between 0.5 and 2 mol/liter of organic solvent.
